(19) United States
(12) Patent Application Publication
(10) Pub. No.: US 2009/0221012 A1
(43) Pub. Date: Sep. 3, 2009

(54) Method for Detecting Procoagulant Phospholipid

(75) Inventor: Thomas EXNER, Gordon (AU)

Correspondence Address: SUGHRUE MION, PLLC, 2100 PENNSYLVANIA AVENUE, N.W., SUITE 800, WASHINGTON, DC, 20037, US

(73) Assignee: Haematex Research Pty Limited, Hornsby (AU)

(21) Appl. No.: 12/391,877

(22) Filed: Feb. 24, 2009

Related U.S. Application Data
(62) Division of application No. 11/384,258, filed on Mar. 21, 2006.
Application No. 11/384,258 is a continuation of application No. PCT/AU2004/001291, filed on Sep. 22, 2004.

(30) Foreign Application Priority Data
Sep. 22, 2003 (AU) 2003-905172

(51) Int. Cl. C12Q 1/56; C12Q 1/02; C12N 9/16

(52) U.S. Cl. 435/13; 435/29; 435/196

(57) ABSTRACT

The present invention relates to a method for determining the amount of procoagulant phospholipid in a sample, said method comprising steps (i) to (iii) performed in the following order: (i) forming an admixture of the sample and a substrate plasma which has been rendered free or substantially free of procoagulant phospholipid sufficient to at least reduce the capacity of the substrate plasma to coagulate, wherein said substrate plasma has been rendered free or substantially free of procoagulant phospholipid by treatment with a phospholipase; (ii) contacting the admixture with a reagent for activating coagulation of plasma in conditions were procoagulant phospholipids is the rate limiting component of the mixture; and (iii) determining the clotting time if the admixture.

### CROSS-REFERENCE TO RELATED APPLICATIONS

This is a division of application Ser. No. 11/384,258 filed Mar. 21, 2006 which is a continuation of International Application No. PCT/AU2004/001291 filed on Sep. 22, 2004, claiming priority based on Australian Application No. 2003-905172 filed on Sep. 22, 2003, the contents of all of which are incorporated herein by reference on their entirety.

### TECHNICAL FIELD

This invention relates to blood coagulation tests and more particularly relates to an improved method for a marker of thrombosis and platelet activation and a potential thrombotic risk factor.

### BACKGROUND ART

Procoagulant phospholipids, including, for example, anionic phospholipids such as phosphatidyl serine, have an important role in the blood coagulation mechanism. Procoagulant phospholipids are required in the intrinsic coagulation pathway for conversion of factor X to Xa by factors VIIIa and IXa and also in the common pathway for cleavage of prothrombin to thrombin by factor Xa. They form part of the tissue factor activator complex. In antithrombotic mechanisms they are involved in the activation of protein C by the thrombin/thrombomodulin complex and in the destruction of factor Va by activated protein C.

Low levels of procoagulant phospholipids are typically present in the blood of healthy individuals, probably as microparticles derived from a variety of cells, principally platelets, but these levels increase when platelets become activated, for example, in response to injury and activation of the blood clotting, complement or immunologic mechanisms. In vitro platelets express maximal procoagulant activity after freeze thawing or activation by collagen/thrombin or membrane disrupting agents such as ionophores. Abnormal activation of platelets in vivo occurs during thrombotic episodes, embolism, sepsis, disseminated intravascular coagulation and infarction. Conversely inadequate activation of platelets occurs in certain bleeding disorders such as von Willebrands disease and with various platelet abnormalities.

Procoagulant phospholipids may be traditionally detected in a sample of patient's blood plasma by a coagulation assay, for example, the Russell's Viper Venom Test (hereinafter “RVVT”), although such assays are more conventionally used for diagnosing lupus anticoagulant. The venom used in the RVVT contains metalloproteases that specifically activate factors V and X. After the addition of venom and calcium ions, coagulation proceeds with a near absolute dependence on procoagulant phospholipid in the patient's sample. The amount of procoagulant phospholipid in the patient's sample is determined according to the time required for the test mixture to form fibrin and coagulate and thereby cease to flow in a tube or increase in optical turbidity or block a hole or aperture. The clotting time or time required for a fibrin clot to form may be replaced as an endpoint indicator in this and subsequent descriptions by a chromogenic substrate which yields a readily-detectable coloured product when acted on by the main clotting enzyme, thrombin.

Where a patient is suspected of having a factor deficiency such as insufficient Factor X, V, II or fibrinogen, or is receiving anticoagulant, the patient's sample is typically mixed with a sample of normal human platelet free plasma for the purpose of supplying those factors which are deficient in the sample. This normal human platelet free plasma is typically known as ‘substrate plasma’. The substrate plasma used in these assays is ideally platelet free otherwise coagulation will not be absolutely dependent on procoagulant phospholipid contained in the patient's sample.

In RVVT and other coagulation assays, substrate plasma is usually prepared by high speed centrifugation and/or filtration. A principal disadvantage of this procedure is that it is difficult to control the depletion of procoagulant phospholipid from the substrate plasma. Fresh plasma is essential and this is often inconvenient to obtain. Once plasma has been frozen, platelets contained therein are activated and release procoagulant phospholipid. Accordingly, the sensitivity provided by RVVT and other coagulation assays for detection of procoagulant phospholipid in the patient's sample, and the capacity to regulate the specificity of these assays is limited. A further disadvantage is that these processes do not remove some cellular microparticles which may have neutral buoyancy or may be too small to be filtered out.

Another disadvantage of current methods for procoagulant phospholipid determination is their sensitivity to coagulation inhibitors, such as antibodies. These antibodies occur frequently in autoimmune disease, eg. “antiphospholipid syndrome”, and cause prolongation of most clotting tests which employ phospholipid-containing reagents and thus give false negative results in current tests for procoagulant phospholipids.

### SUMMARY OF THE INVENTION

In view of the role of procoagulant phospholipids in the pathogenesis of thrombotic episodes and their potential as markers of platelet or cellular activation, there is a need for an improved method for detecting the presence of and the amount of procoagulant phospholipid in a sample.

Therefore, according to a first aspect of this invention there is provided a method of determining the amount of procoagulant phospholipid in a sample, the method comprising steps (i) to (iii) performed in the following order: (i) forming an admixture of the sample and a substrate plasma which has been Tendered free or substantially free of procoagulant phospholipid sufficient to at least reduce the capacity of the substrate plasma to coagulate, wherein said substrate plasma has been rendered free or substantially free of procoagulant phospholipid by treatment with a phospholipase; (ii) contacting the admixture with a reagent for activating coagulation of plasma in conditions where procoagulant phospholipid is the rate limiting component of the mixture; and (iii) determining the clotting time of the admixture.

According to a second aspect of this invention there is provided a method of determining the amount of activated platelets and cell derived microparticles in a sample, the method comprising steps (i) to (iii) performed in the following order: (i) forming an admixture of the sample and a substrate plasma which has been rendered free or substantially free of procoagulant phospholipid sufficient to at least reduce the capacity of the substrate plasma to coagulate; (ii) contacting the admixture with a reagent for activating coagulation of plasma in conditions for permitting procoagulant phospholipid to coagulate the admixture; and (iii) determining the clotting time of the admixture.

According to a third aspect of this invention there is provided a method of assessing whether a patient has had a recent thrombotic episode, the method comprising steps (i) to (iii) performed in the following order: (i) forming an admixture of the sample and a substrate plasma which has been rendered free or substantially free of procoagulant phospholipid sufficient to at least reduce the capacity of the substrate plasma to coagulate; (ii) contacting the admixture with a reagent for activating coagulation of plasma in conditions for permitting procoagulant phospholipid to coagulate the admixture; and (iii) determining the clotting time of the admixture.

A thrombotic episode for example may be deep vein thrombosis, embolism or infarction. By “recent” is meant within the time limit that procoagulant phospholipid derived from the thrombotic event may be detected in the circulation. An estimate would be up to 12 hours from such an event if no further platelet activation occurs.

According to a fourth aspect of this invention there is provided a method of producing a substrate plasma for use in determining the level of procoagulant phospholipid in a sample, said method comprising treating substrate plasma with a phospholipase for degrading procoagulant phospholipid sufficient to at least reduce the capacity of the substrate plasma to coagulate.

According to a fifth aspect of this invention there is provided a substrate plasma produced by the method of the fourth aspect. This includes the concept of incubating a test plasma containing an unknown amount of procoagulant phospholipid alone with phospholipase and comparing the result of a phospholipid-sensitive test before and after such an incubation. A significant prolongation of the test confirms that procoagulant phospholipid had been present without any need for addition of phospholipid free substrate plasma.

According to a sixth aspect of this invention there is provided a kit for determining the level of procoagulant phospholipid in a sample, said kit comprising: (i) a substrate plasma which has been treated with a phospholipase for degrading phospholipid sufficient to at least reduce the capacity of the substrate plasma to coagulate; (ii) a reagent for activating coagulation of plasma in a phospholipid-dependent manner; and (iii) reference preparations containing known levels of procoagulant phospholipid.

The reference preparations containing known levels of procoagulant phospholipid may be used as calibrating agents to construct a reference graph.

### DISCLOSURE OF THE INVENTION

The invention seeks to address the disadvantages identified above and in one embodiment provides a method for determining whether a sample contains detectable procoagulant phospholipid above the lower sensitivity limit of the method and in a second embodiment, how much. The method comprises forming an admixture of the sample and a substrate plasma which has been rendered free or substantially free of procoagulant phospholipid sufficient to at least reduce the capacity of the substrate plasma to coagulate in a phospholipid-dependent clotting test. The substrate plasma may be rendered free or substantially free of procoagulant phospholipid by treatment with a phospholipase.

The phospholipid-dependent clotting test may be one that is initiated by Russells viper venom or the factor X activator from that venom or the phospholipid dependent prothrombin activator from Pseudonaja Textiis venom or more preferably factor Xa of human, animal or recombinant origin.

The plasma may be human plasma or non-human plasma and is preferably non-human plasma and more preferably animal plasma.

The plasma may be rendered free or substantially free of procoagulant phospholipid by for example, treating with an enzyme which degrades phospholipid in the plasma.

For example to prolong the factor Xa activated clotting time of horse plasma from 50 to 120 sec requires 1 hour incubation at 37° C. with 2×10⁻⁵% Naja nigricollis venom. Details of various plasma pre-treatment protocols are shown in Example 1 below. The admixture is then contacted with a reagent for activating coagulation of plasma in conditions where the concentration of procoagulant phospholipid influences the clotting time. A determination as to whether the sample comprises procoagulant phospholipid is made by determining when coagulation of the admixture has occurred.

As described herein, the inventor has found that the sensitivity of a clotting test for detecting procoagulant phospholipid in a sample, such as preferably with a factor Xa-based test, is improved by using as a substrate plasma, a composition in which procoagulant phospholipid has been degraded by treatment with phospholipase. More specifically, an admixture comprising a substrate plasma treated with phospholipase was observed to have an increased clotting time, relative to the clotting time of an admixture comprising untreated platelet poor plasma or normally-treated centrifuged plasma. (Example 2). As the same amounts of test plasma and therefore, the same amounts of added procoagulant phospholipid were provided in all admixtures, it follows that the decreased clotting time in the admixture comprising non-treated and centrifuged substrate plasmas was caused by detection of procoagulant phospholipid from both the substrate plasma and the sample. The admixture comprising the treated substrate plasma, in having an increased clotting time, has improved sensitivity because the only procoagulant phospholipid contributed to the admixture and therefore, which is detected in the assay, is derived from the sample.

The results are surprising because many enzymes typically are not capable of activity when added to plasma. This is because plasma is a complex mixture of heterogenous molecules which can prevent enzyme activity. For example, plasma contains proteins which strongly bind to phospholipids such as apolipoproteins, annexins and beta-2-glycoprotein 1 and these may interfere with the availability of substrate for a phospholipase. Further, phospholipases usually require calcium for their enzymatic activity and this is greatly reduced by the citrate anticoagulant normally used in plasma collected for blood clotting tests. Further, plasma also comprises inhibitor molecules capable of inhibiting the activity of specific enzymes. For example, antitrypsin which binds to and inhibits trypsin, antithrombin which inhibits thrombin and antiplasmins which inhibit plasmin activity. Probably the main inhibitor of most phospholipases in human plasma is annexin V.

Typically the substrate plasma is one which has been treated with a phospholipase. An example of such a phospholipase is a basic phospholipase A2. The phospholipase may be produced synthetically, for example by recombinant DNA technology, or may be derived from an organism. For example, the phospholipase may be derived from snake venom. As exemplified herein, phospholipases derived from the venom of Naja mossambica and N nigricollis are particularly useful for treating the substrate plasma. Other types of venom which are useful are derived from Agkistrodon halys, Vipera species, especially Berus and Russelli, Crotalus durissus, Enhydrina schistosa, Oxyuranus scutellatus and Apis melifera. The main characteristic of venom phospholipases which makes them effective in plasmas is probably their basic character as shown by a high isoelectric pH. Most of the effective venom-derived phospholipases share structural homology.

Other organisms which may provide a phospholipase for use in treating the substrate plasma include Streptomyces violaceoruber, Vibrio species, Clostridium perfringens, Bacillus cereus.

It follows that as anionic phospholipids, such as phosphatidyl serine are important in thrombosis, typically the enzyme for degrading the procoagulant phospholipid in the substrate plasma should be one capable of degrading phosphatidyl serine in plasma. As noted above, the use of a substrate plasma treated accordingly improves the specificity for detection of phosphatidyl serine in a coagulation assay for detection of procoagulant phospholipid, such as RVVT or factor Xa-based test.

It is to be understood that the substrate plasma for use in the method of the invention does not need to be treated to degrade all phospholipid in it. However the substrate plasma is typically treated so that its capacity to coagulate, when activated by a procoagulant phospholipid-dependent activator of coagulation, for example Russell's Viper Venom, is at least reduced by the degradation of procoagulant phospholipid in the substrate plasma by the enzyme. Typically, the capacity of the substrate plasma to coagulate when activated by such a reagent is reduced when substantially all of the procoagulant phospholipid, mainly phosphatidyl serine component of the phospholipid in the substrate plasma, has been degraded by the enzyme. The treatment of the substrate plasma with 1×10⁻⁵% of a whole N nigricollis venom (containing substantially less of the purified enzyme) for about 1 hour at about 37° C. is typically sufficient for degrading substantially all of the procoagulant phospholipid in platelet poor substrate plasma by the enzyme. The actual conditions for depleting individual plasmas depends strongly on their initial content of free procoagulant phospholipid and this depends in turn on the degree of contamination by platelets or other cellular debris (eg see Example 1). Thus plasmas containing high levels of platelets require a longer incubation time or a higher concentration of phospholipase than those which are already low in phospholipid. It is preferable to begin with plasmas which are already low in phospholipid. This phospholipase treatment degrades only about 0.001% of the total 0.1% phospholipid in most platelet poor plasmas. Typically, the proportion of free phosphatidyl serine: total phospholipid is about 1:100,000. A phospholipid-sensitive test such as the factor Xa-activated clotting time (hereinafter “XACT”) routinely detects 100-1000 ng/mL in patient plasmas. It will be understood that a shorter incubation time could be used with a higher concentration of phospholipase and a longer incubation time would be needed with a lower concentration of phospholipase. Thus, 400 ng/mL of N nigricollis venom (NNV) in normal porcine plasma requires 40 minutes at 37° C. to prolong a factor X activated clotting time from 48 sec to 100 sec whereas 200 ng/mL NNV requires 90 minutes to achieve a similar 100 sec optimal XACT result (See example 1 for more details). It will also be understood that the method of the invention will be most sensitive for procoagulant phospholipid when all procoagulant phospholipid in the substrate plasma has been degraded by treatment with the enzyme.

Because the activity of venoms useful in this invention is progressive in nature it is desirable to stop their interaction with plasma once the phospholipid level has been depleted adequately. This may be done with dilute antisera and antibodies directed against the venom being used. Commercially available antivenoms against the particular class of venom, eg cobra, being used are effective at concentrations from 0.01 to 1%.

The substrate plasma can be any composition which corrects for a factor or factors that the patient's sample is deficient in. For example, where the patient's sample is deficient in Factor V, the substrate plasma would contain excess Factor V so as to be capable of effecting coagulation of the patient's sample of plasma. Another example of a substrate plasma is one which contains all factors selected from the group consisting of: factor XII, prekallikrein, high molecular weight kininogen, factor XI, factor VIII, factor IX, factor X, factor V, prothrombin, and fibrinogen at functional levels sufficient to compensate for any defects in the admixed sample. Such a substrate plasma would be used in a kaolin or surface-activated clotting time test. When a test employing tissue factor is used as an activator the substrate plasma must contain factors VII, X, V, II and I (fibrinogen) for the same purpose.

When a test such as the Russell's Viper Venom Test is to be used, this requires only coagulation factors X and below in the coagulation cascade to be present, ie factors X, factor V, factor II and fibrinogen. Factors above factor X need not be present for a normal result. If a factor Xa-based test is to be used, even factor X is not necessary in the system for a normal result, only factors V, II and I (fibrinogen) are then required. The phospholipid-dependent prothrombin activators from elapidae venoms require no factors above factor II (prothrombin) to induce clotting. Thus, if a Taipan venom-based test were to be used only prothrombin and fibrinogen need be provided for a normal result. Fibrinogen or factor I is necessary only to provide a marker for a clotting endpoint. The maximum rate of thrombin generation can be alternatively detected using chromogenic tripeptide substrates which are converted by thrombin to coloured end-products which can be detected spectrophotometrically.

Typically the substrate plasma is derived from citrated blood. Suitable plasmas are those which are known to be effective in promoting coagulation of a human plasma sample, because they provide a factor variably present in the test sample. Examples of such plasmas include most mammalian plasmas. Those which are exemplified herein to be useful in the method of the invention include plasma derived from pig, horse, cow, sheep, goat, camel, monkey, dog, cat, fox, elephant, llama, rabbit, mink, racoon, kangaroo, human and mixtures thereof.

The plasma for providing the substrate plasma may be derived from the individual who is being tested for presence and/or amount of procoagulant phospholipid. In this case the plasma specimen can be tested with a factor Xa activated clotting time before and after incubation with a known amount of phospholipase (eg 100 ng/mL of N nigricollis venom). The difference between the first and second results is proportional to how much procoagulant phospholipid was destroyed by the phospholipase treatment.

However, as antibodies are generated in some humans which have serological activity against human proteins which bind to procoagulant phospholipids, such as beta 2 glycoprotein 1 and prothrombin, (for example lupus inhibitor antibodies), the use of human plasma as a substrate plasma in the method of the invention carries with it some unwanted sensitivity to such inhibitors. Consequently such specimens should be assayed for the presence of these antibodies. Where animal plasma is used to provide the substrate plasma, an advantage of the invention is that the method is much less sensitive to antibodies directed against human clotting factors or lupus cofactors than a method based on human plasma. Such antibodies can occur unexpectedly among patients causing confusion and unreliability from existing clotting test methods.

It will be understood that when a test specimen has altered coagulability, particularly where the individual to be tested has been administered with an anti-coagulant, it may be necessary for the substrate plasma to further comprise at least one agent for controlling the capacity of the anti-coagulant to inhibit coagulation. Those agents which are most likely to be useful are ones capable of controlling the capacity of heparin to inhibit coagulation, because heparin is widely used as an anti-coagulant. These agents include protamine sulphate and polybrene or protamine sulphate. However, other agents include antibodies against hirudin and its analogues or other anticoagulant antagonists.

The substrate plasma would normally be used in a liquid or reconstituted form. However for use in a “point of care” device it could be present as part of a dry composition reconstituted by the applied specimen of blood or plasma itself.

The reagent for activating coagulation of the admixture in the test must activate coagulation to proceed subsequently in a procoagulant phospholipid-dependent manner. Examples of such reagents are those capable of converting factor X to factor Xa, or capable of converting prothrombin to thrombin. Accordingly, the reagent for use in the method of the invention may be Russell's Viper Venom or factor X activator from a related venom of the viperidae family or factor Xa or other phospholipid-dependent prothrombin activator derived from elapid venoms such as the Australian cobra Pseudonaja or Oxyuranus scutellatus family. Reagents derived from mammals other than human are particularly useful, for example factor Xa of bovine origin (see Example 4). Reagents acting higher up the coagulation mechanism such as contact activators, tissue factor, factor IXa, factor XIa and factor VIIa can be used, but these make the system less specific for phospholipid and more vulnerable to interference by patient plasma variables.

Clotting activators may also be enzymes from recombinant precursors based on novel DNA sequences. Such procoagulants could be rendered insensitive to inhibiting antibodies by deletion of common epitopes recognised by such antibodies. These reagents would normally be used in liquid form but could also be provided in a dried form for application in a “point of care” device, in which case they would be reconstituted by an applied specimen of plasma or blood specimen.

While it is anticipated that the method of the invention would be most widely applied in relation to a plasma or blood sample derived from a human patient, it is to be understood that the method can be used to detect procoagulant phospholipid in a range of animals. This embodiment would be useful in animal experimental studies for in vivo or in vitro assessment of the biocompatability of materials' surfaces with animal blood and the effect of experimental drugs. The sample to be tested for procoagulant phospholipid can be blood, plasma, serum or any other fluid. If anticoagulated by calcium-binding agents such as citrate or EDTA, the levels of such agents should be similar to those used in other clotting tests.

In the first aspect of the invention mentioned above, there is provided a method of determining the amount of procoagulant phospholipid in a sample.

The measurement is made in comparison with reference plasmas containing known amounts of procoagulant phospholipid and unknown values may be interpolated from an appropriately constructed calibration curve.

As procoagulant phospholipids are typically located on activated platelets and platelet microparticles, it follows that measuring the amount of procoagulant phospholipid in platelet rich plasma according to the first aspect of the invention would enable one to quantitate the amount of activated platelets and platelet microparticles in the sample.

Thus in the second aspect mentioned above, the invention provides a method of determining the amount of activated platelets and cell-derived microparticles in a sample, the method according to the first aspect of the invention.

As noted above, abnormal platelet or cellular activation may result from thrombotic episodes, embolism, tissue trauma, immune processes (including complement activation), sepsis, disseminated intravascular coagulation or infarction. In extreme cases, or when due to immunologic processes it can result in thrombocytopenia. It would be advantageous to be able to determine whether an individual has a clinical condition involving platelet activation, for example, thrombosis, stroke or myocardial infarction. The inventor recognises that a method for determining the amount of activated platelets or platelet microparticles, by determining the amount of procoagulant phospholipid would allow diagnosis of those individuals with those conditions.

Thus in the third aspect mentioned above, the invention provides a method of assessing whether a patient has recently had a thrombotic episode such as a deep vein thrombosis, embolism, infarction, the method according to the second aspect of the invention.

In the fourth aspect mentioned above, the invention provides a method for producing a substrate plasma for use in determining whether an individual comprises procoagulant phospholipid. The method comprises treating substrate plasma with an enzyme for degrading procoagulant phospholipid sufficient to at least reduce the capacity of the substrate plasma to coagulate.

Using an enzyme in the method of the fourth aspect of the invention, one is able to provide a panel of substrate plasmas comprising defined amounts of procoagulant phospholipid. This allows one to control the sensitivity of the methods of the first and second aspect of the invention, by selecting for use from the panel, a substrate plasma comprising the desired amount of procoagulant phospholipid. This option provides a reasonable or optimised baseline clotting time for a particular instrument. Snake venoms are particularly useful to provide an enzyme for use in the fourth aspect of the invention because they can be used at very low concentrations and their activity can be controlled subsequently for example, by the use of antisera and antibodies effective against phospholipases. However, it will be understood that agents capable of controlling phospholipase enzymes derived from recombinant DNA technology, or from other organisms or inhibitory compounds could be used. Thus, a further step of the method of the fourth aspect of the invention comprises contacting the substrate plasma with at least one agent for controlling the capacity of the enzyme to degrade procoagulant phospholipid.

Also, in another embodiment, the method of the fourth aspect comprises the further step of mixing the substrate plasma with at least one agent such as Polybrene or protamine sulphate for controlling the capacity of a therapeutic anticoagulant such as heparin to inhibit coagulation.

In the fifth aspect mentioned above, the invention provides a substrate plasma produced by the method of the fourth aspect.

In the sixth aspect mentioned above, the invention provides a kit for determining whether an individual comprises procoagulant phospholipid, the kit comprising: (i) a substrate plasma which has been treated with an enzyme for degrading phospholipid sufficient to at least reduce the capacity of the substrate plasma to coagulate; and (ii) a reagent for activating coagulation of plasma in a phospholipid-dependent manner (iii) reference preparations containing known levels of procoagulant phospholipid, wherein the reference preparations containing known levels of procoagulant phospholipid may be used as calibrating agents to construct a reference graph.

### BRIEF DESCRIPTION OF THE DRAWINGS

The invention is further described with reference to the drawings in which:

FIG. 1 is a representation of the effect of incubating normal plasmas from various species with or without N nigricollis venom as described in Example 1;

FIG. 2 is a representation of the effect of pretreatment with N nigricollis venom on platelet sensitivity; and

FIG. 3 is a representation of the sensitivity of various tests for platelet phospholipid.

### BEST MODES AND OTHER MODES FOR CARRYING OUT THE INVENTION

The present invention will now be described with reference to the following examples which should not be construed as limiting on the scope thereof.

### Example 1

### Progressive Effect of N nigricollis Venom on Crude Animal Plasmas

Aim: To demonstrate the progressive and selective effect of a typical venom phospholipase in reducing the procoagulant phospholipid from platelet-containing plasmas from various species, thereby improving the sensitivity of those substrate plasmas in clotting tests for procoagulant phospholipid.
Method: Blood samples were collected into one tenth its final volume of 3.2% trisodium citrate anticoagulant by clean venipuncture from a human volunteer, by cardiac puncture from a freshly shot horse (equine), by an arterial bleed from a pig at an abattoir and similarly from an ox (bovine). The samples were centrifuged at 3,000 rpm for 20 minutes and the supernatant platelet poor plasmas with quite variable platelet counts (approximately 5×10⁹/L for the human sample, but not measured for the animal plasmas) were frozen at −30° C.

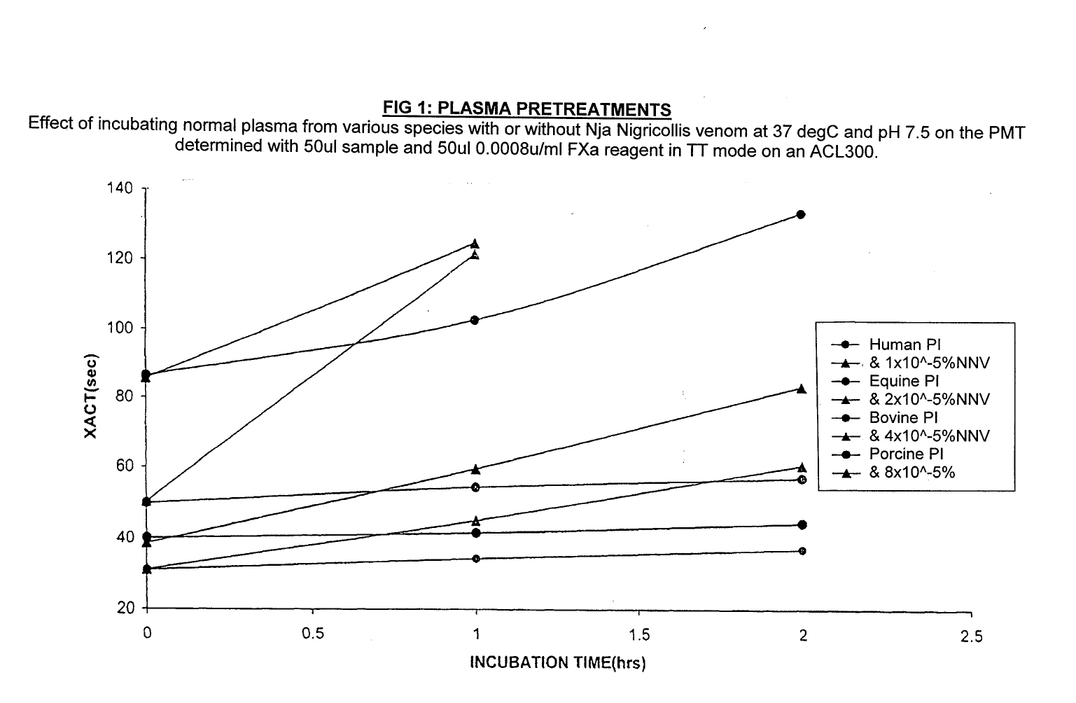

Subsequently thawed platelet poor samples were incubated at 37° C. without treatment or after mixing with N nigricollis venom (NNV) at the concentrations shown in FIG. 1 Specimens were removed at 1 or 2 hour intervals and tested with a factor Xa/calcium reagent for the XACT test.

Results: These are shown in FIG. 1 XACT results on all plasmas without NNV additions were reasonably stable. With NNV present however XACT results prolonged over the incubation period.
Bovine and porcine plasmas gave the shortest initial results, probably due to excess free procoagulant phospholipid, but these both doubled after incubation for 2 hours with 4×10⁻⁵% and 8×10⁻⁵% NNV. The XACT on horse plasma prolonged from 50 to 120 sec after 1 hour with 2×10⁻⁵% NNV.
Comments: These increases in XACT results due to incubation with NNV were not accompanied by significant changes in activated partial thromboplastin time (APTT), prothrombin time (PT) or other clotting tests. This confirms that the major effect of the NNV was not due to degradation of coagulation factors involved in these clotting tests, but rather to loss of phospholipid for which these tests are not sensitive.

### Example 2

### Pretreatment of Human Plasma with N nigricollis Venom

Aim: To show that treatment of a normal human plasma with a trace of N nigricollis venom gives a product (substrate plasma) with better sensitivity to platelets in a Factor Xa-based clotting test than centrifugation.
Method: Test plasmas containing varying levels of freeze-thawed normal platelet rich plasma (PRP initially with 250×10⁹ platelets/L) in platelet “free” normal human plasma were prepared. The platelet free plasma (PFP) was obtained by high speed centrifugation and filtration through a 0.22 micron syringe filter.

These test plasmas were mixed with an equal volume of 3 different substrate plasmas before being tested in a factor Xa-based clotting test. The 3 different substrate plasmas were:

1. Normal platelet “poor” human plasma (PPP).

2. The same PPP centrifuged at 15,000 g for 10 min.

3. The same PPP treated with 1×10⁻⁵% N nigricollis venom for 20 minutes at 37° C. (hereinafter the “NNV treatment”).

The factor Xa reagent contained 0.001 U/mL bovine Factor Xa in 0.015 M calcium chloride, 0.1M sodium chloride, 0.02M HEPES pH 7.0 buffer and was used in a proportion of 0.05 mL plasma mix with 0.1 mL of reagent in a ST4 (Diagnostica Stago, Paris, France) clotting machine at 37° C.

Results: Table 1 shows Factor Xa clotting time results in seconds on 1:1 mixes of test plasmas containing various platelet counts and substrate pooled normal plasma (PNP) pretreated by the two different methods.

TABLE 1

| Test Plasmas | Substrate Plasmas | | |
|---|---|---|---|
| Platelet count | | Centrifuged | PNP after NNV |
| (10⁹/L) | PNP initially | PNP | treatment |
| 25 | 48.2 | 45.9 | 50.8 |
| 5 | 58.9 | 66.2 | 73.9 |
| 1 | 64.1 | 85.7 | 96.4 |
| <0.2 (PFP) | 67.7 | 95.4 | 117 |

Comment: These experiments show that NNV treatment achieved a greater increase in clotting time results over those obtained with high speed centrifugation. This resulted in an improvement in sensitivity to platelets.

### Example 3

### Effect of a Pre-Treatment with N nigricollis Venom on Platelet Sensitivity

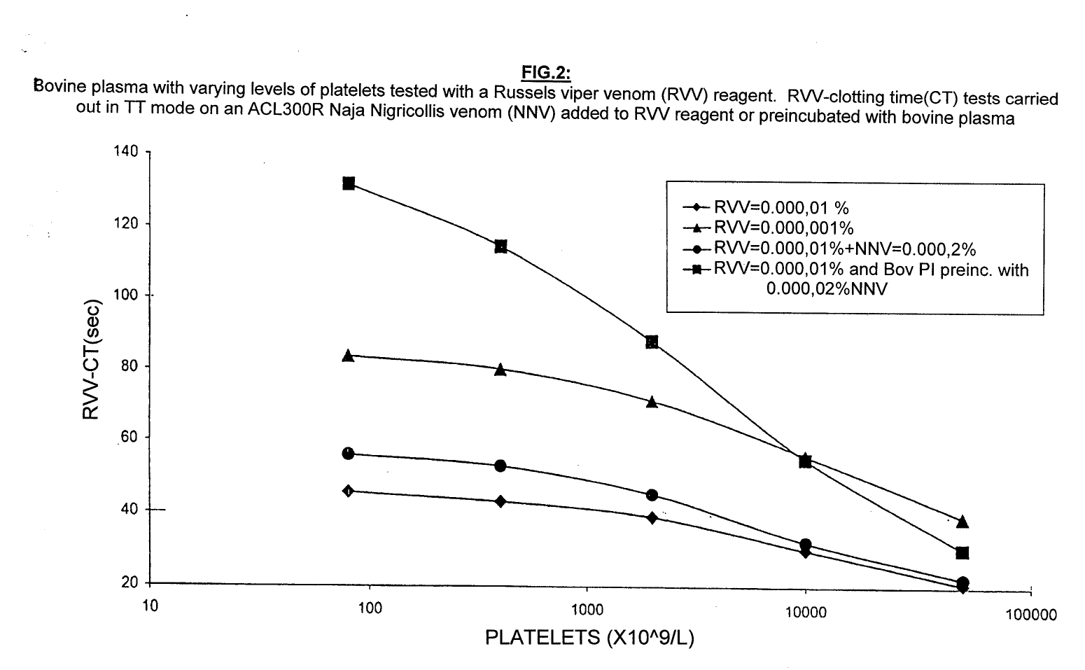

Aim: To demonstrate the effect of N nigricollis venom in enhancing the sensitivity of a Russells viper venom clotting test system based on bovine plasma.
Method: A series of dilutions of a frozen-thawed, though otherwise normal human platelet rich plasma (with initial platelet count of 250×10⁹/L) were made in normal bovine plasma and also in bovine plasma pretreated for 50 min at 20° C. with 5×10⁻⁵% N nigricollis venom. These plasma samples were mixed with an equal volume of various Russell's viper venom and calcium-containing reagents and timed to a clotting endpoint at 37° C. in thrombin time mode (TT mode uses equal volumes of plasma and reagent) in a ACL300 clot-timing instrument (Instrumentation Laboratory SpA, Milan, Italy). The Russell's viper venom concentration in the reagent with 0.025M calcium chloride was varied from 10⁻⁵% to 10⁻⁶% and the former reagent was also tested after the addition of 2×10⁻⁴% N nigricollis venom.
Results: The results obtained are summarised in FIG. 2. It is apparent that the sensitivity of a test system using 1×10⁻⁵% RVV to platelets was quite low, plateauing out at platelet levels below 1×10⁹/L. RVV clotting times were prolonged by reducing the RVV concentration tenfold to 10⁻⁶%, but sensitivity to platelets as shown by the gradient of the responsiveness curve was not improved. Including 20×10⁻⁵% NNV in the RVV reagent (RVV=10⁻⁵%) increased the sensitivity slightly.

The highest sensitivity to platelets was observed when the bovine plasma had been preincubated with 5×10⁻⁵% NNV before being used to dilute out the platelet concentrate. In this case platelet counts between 0.1 and 1.0 could still be quantitated accurately.

### Example 4

### Comparison of Various Clotting Activators

Aim: To compare the sensitivities of 4 different phospholipid-dependent clotting activators in a test system for assaying procoagulant phospholipid.
Method: Dilutions of a platelet rich plasma were prepared in platelet free normal human plasma as shown below. These samples were tested with 4 different clotting test systems. All tests were carried out at 37° C. in a ST4. The reagents and methods were as follows.

1. Kaolin clotting tests (KCT) were carried out using 0.05 mL plasma samples preincubated with 0.05 mL of 1% kaolin suspension in water for 3 min and then recalcified with 0.05 mL of 0.025 M calcium chloride. The time from addition of calcium chloride till clotting occurred was determined in a ST4 (Stago) clotting machine.

2. Russell's Viper Venom Tests (RVV) were carried out by mixing 0.05 mL samples with 0.05 mL of a reagent containing 2×10⁻⁶% RVV in 0.025M calcium chloride and timing till a clotting endpoint.

3. Factor Xa-based clotting tests (FXa-CT) were carried out by mixing 0.05 mL samples with 0.05 mL of a reagent containing 0.001 U/mL bovine factor Xa in 0.025 M calcium chloride and timing to a clotting endpoint.

4. Textarin (TM-Pentapharm, Basel, Switzerland) clotting tests (TX-CT) were carried out by mixing 0.05 mL samples with 0.05 mL of a reagent containing 2 U/mL of delipidated commercial Textarin reagent in 0.025 M calcium chloride and timing to a clotting endpoint.

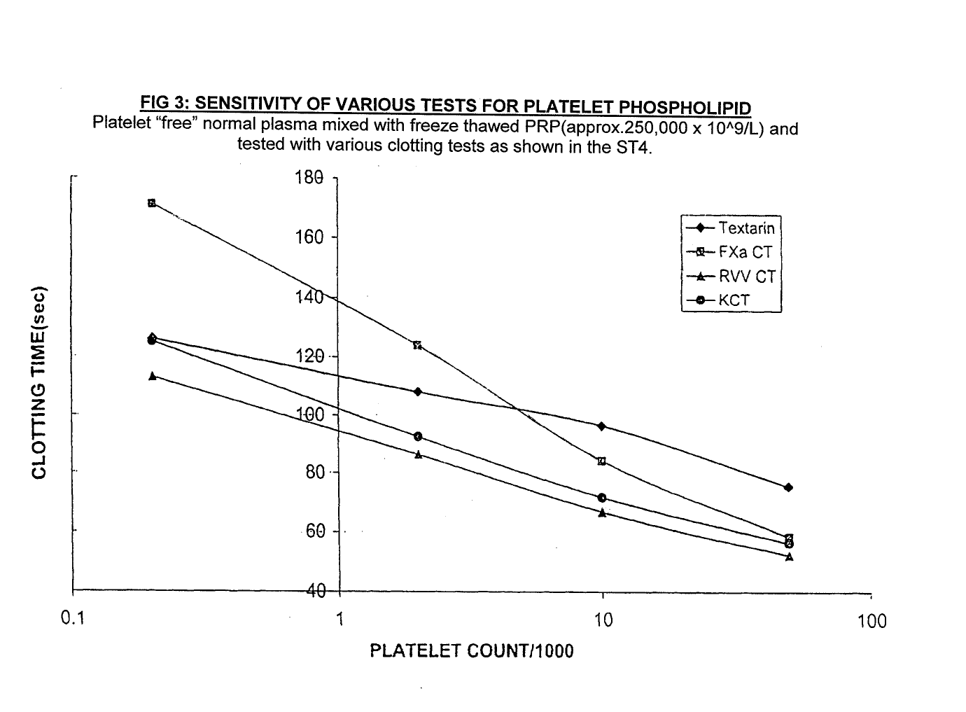

Results: Results obtained are shown in FIG. 3. The RVVT and KCT tests showed similar sensitivities to platelets. The clotting test based on activated factor X (XACT) showed the highest sensitivity to platelets. The test with the lowest sensitivity to platelets was that based on delipidated Textarin. However it is possible that this may have been due to inadequate removal of phospholipid from this commercial reagent intended for an alternative purpose, ie. detection of lupus inhibitors.
Comments: The Textarin reagent is a typical phospholipid-dependent prothrombin activator as derived from the venom of Pseudonaja textiles, one of several Australian elapids known to contain such procoagulants.

### Example 5

### Typical Use of the Method and Specificity Study

Aim: To illustrate that the method is insensitive to defects in all known clotting factors. Also to detect free procoagulant phospholipid in various commercially-available plasmas deficient in individual clotting factors.
Method: Various freeze-dried individual clotting factor deficient plasmas marketed for use in specific factor assays were tested using the new test for procoagulant phospholipid. Thus the vials from various suppliers (Dade/Behring, IL/Beckman-Coulter and Diagnostica Stago) were each freshly reconstituted with 1 mL of water. The tests used 25 μl of NNV-treated substrate plasma (lot 3004) with 25 μL of each factor deficient plasma and 50 μl of factor Xa reagent in a Stago ST4 clotting machine.
Results: These are shown in Table 2.

TABLE 2

| Test Plasma | FXa-Clotting Time(Sec) |
|---|---|
| Frozen“platelet-poor” plasma | 53.7 |
| Platelet “free” normal plasma | 102 |
| Prothrombin(FII) deficient | 88.8 |
| Factor V deficient plasma | 100 |
| Factor VII deficient | 96.5 |
| Factor VIII deficient | 70.0 |
| Factor IX deficient | 71.6 |
| Factor X deficient | 73.7 |
| Factor XI deficient | 88.3 |
| Factor XII deficient | 96.7 |

Comment: The pooled frozen platelet-poor plasma gave a relatively short FXa clotting time compared with the platelet free normal plasma because it contained approximately 5% of a normal platelet count (approximately 10×10⁹ platelets/L).

These results show that the total deficiency of any individual plasma clotting factor in a test sample does not prolong the FXa test. It also shows that the factor VIII, IX and X deficient plasmas used here contain appreciable amounts of procoagulant phospholipid detectable with this test.

### INDUSTRIAL APPLICABILITY

It should be clear that the methods of the present invention will find wide application in clinical laboratory science.

The foregoing describes only some embodiments of the present invention and modifications obvious to those skilled in the art can be made thereto without departing from the scope of the invention.

1. A method for determining the amount of activated platelets and cell-derived microparticles in a sample, said method comprising steps (i) to (iii) performed in the following order:
(i) forming an admixture of the sample and a substrate plasma which has been rendered free or substantially free of procoagulant phospholipid sufficient to at least reduce the capacity of the substrate plasma to coagulate;
(ii) contacting the admixture with a reagent for activating coagulation of plasma in conditions for permitting procoagulant phospholipid to coagulate the admixture; and
(iii) determining the clotting time of the admixture.

2. A method according to claim 1 wherein said method determines if a patient has had a recent thrombotic episode.

3. A method according to claim 1 wherein said method determines if a patient has had a clinical disorder involving platelet activation.

4. The method according to claim 2 wherein the thrombotic episode is selected from the group consisting of disseminated intravascular coagulation, deep vein thrombosis, embolism, tissue trauma, sepsis, and infarction.

5. The method of claim 1 wherein said substrate plasma has been rendered free or substantially free of procoagulant phospholipid by treatment with a phospholipase.

6. The method of claim 5 wherein the phospholipase is obtained from venom selected from the group consisting of Naja mossambica, Naja nigricollis, Agkistrodon halys, Vipera Berus, Vipera Russeli, Crotalus durissus, Enhyrdrina schistose, Oxyuranus scutellatus and Apis mellifera.

7. The method of claim 5 wherein the phospholipase is obtained from one of a selected group consisting of Steptromyces violaceoruber, Vibrio species, Clostridium perfringens, or Bacillus cereus.

8. The method of claim 1 wherein the reagent for activating coagulation of plasma is factor Xa.

9. The method of claim 1 wherein the reagent for activating coagulation of plasma is capable of converting factor X to factor Xa.

10. The method of claim 9 wherein the reagent is Russell's Viper Venom.

11. The method of claim 9 wherein the reagent is a factor X activator from a venom of the viperidae family.

12. The method of claim 1 wherein the reagent for activating coagulation of plasma is capable of converting prothrombin to thrombin.

13. The method of claim 12 wherein the conversion of prothrombin to thrombin is in a phospholipid-dependent manner.

14. The method of claim 12 wherein the reagent is a phospholipid dependent prothrombin activator derived from elapid venoms.

15. The method of claim 14 wherein the elapid venom is from the Australian cobra Pseudonaja or Oxyuranus scutellatus family.

16. The method of claim 1 wherein the substrate plasma has been formed from factors V and prothrombin.

17. The method of claim 16, wherein said factors V and prothrombin are phospholipid free.

18. The method of claim 16, wherein said factors V and prothrombin are of animal or human origin.

19. The method according to claim 5 wherein the substrate plasma of the said method is contacted with at least one agent for controlling the capacity of the enzyme to degrade procoagulant phospholipid.

20. The method according to claim 19, further comprising the step of mixing the substrate plasma with at least one agent for controlling the capacity of a therapeutic anticoagulant to inhibit coagulation.

21. The method according to claim 20 wherein the agent for controlling the capacity of a therapeutic anticoagulant to inhibit coagulation is Polybrene or protamine sulphate.

22. The method according to claim 1 wherein said substrate plasma is obtained from a member selected from the group consisting of pig, horse, cow, sheep, goat, camel, monkey, dog, cat, fox, elephant, llama, rabbit, mink, racoon, kangaroo, human and mixtures thereof.

23. A method of producing a substrate plasma for use in determining the level of procoagulant phospholipid in a sample, said method comprising treating substrate plasma with a phospholipase for degrading procoagulant phospholipid sufficient to at least reduce the capacity of the substrate plasma to coagulate.

24. A substrate plasma produced by the method of claim 23.
